(19) United States
(12) Patent Application Publication
(10) Pub. No.: US 2026/0174937 A1
(43) Pub. Date: Jun. 25, 2026

(54) FIBRIN COMPOSITIONS, SUBSTRATES THEREOF, AND METHODS OF MAKING THEREOF

(71) Applicant: THE UAB RESEARCH FOUNDATION, BIRMINGHAM, AL (US)

(72) Inventors: Jesse Jones, Mountain Brook, AL (US); Vinoy Thomas, Hoover, AL (US)

(21) Appl. No.: 19/429,328

(22) Filed: Dec. 22, 2025

Related U.S. Application Data
(60) Provisional application No. 63/737,931, filed on Dec. 23, 2024.

(51) Int. Cl. A61L 29/08; A61L 29/16; A61L 31/10; A61L 33/00; A61L 33/12

(52) U.S. Cl. CPC A61L 29/085; A61L 29/16; A61L 31/10; A61L 33/0082; A61L 33/126; A61L 2400/18; A61L 2420/02

(57) ABSTRACT

In one aspect, the disclosure relates to medical devices, such as a stent retriever, stint, catheter, comprising coatings. A coating can comprise a fibrin-based material. The fibrin-based material within a coating on a medical device can form covalent bonds with fibrin present in a thrombus for the purposes of a thrombus extraction.

### CROSS-REFERENCE TO RELATED APPLICATIONS

This application claims the benefit of and priority to U.S. Provisional Application No. 63/737,931 filed on Dec. 23, 2024 which is incorporated herein by reference in its entirety.

### BACKGROUND

Ischemic stroke is a major cause of disability and death worldwide. Stent retrievers (SRs) has improved patient outcomes for large and medium vessel occlusions. Such devices are delivered into the occlusive thrombus by an endovascular approach and mechanically “grasp” it, enabling rapid removal. However, there are still limitations to the current technologies.

### SUMMARY

The present disclosure provides for medical devices having a coating, medical devices for the treatment of thromboembolism and other conditions, methods of use of the medical devices, methods for putting a coating on a medical device, and the like

In an aspect, the present disclosure provides for a medical device, comprising: a substrate; and a coating on a surface the substrate, the coating comprising a fibrin-based material, wherein the medical device is a stent, a stent retriever, a catheter (e.g., aspiration catheter), metal wires, or medical plastic tubes.

In an aspect, the present disclosure provides for a method for treating thromboembolism, comprising: contacting a thrombus in a subject with the medical device of as described above and herein; allowing the medical device to capture the thrombus; and optionally removing the thrombus from the subject.

In an aspect, the present disclosure provides for a method for forming a coating on a substrate, comprising: modifying a substrate via plasma polymerization; exposing the substrate to a fibrinogen solution; and forming a coating on a surface of the substrate, wherein the coating comprises a fibrin-based material.

Other devices, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows FT-IR spectra of SR before and after modification with ME plasma (red), fibrinogen (blue), and EDC (green). FT-IR: Fourier transform infrared; SR: stent retriever; ME: mercaptoethanol; EDC: 1-ethyl-3-(3-dimethylaminopropyl) carbodiimide.

FIG. 2A shows OPT-IR spectra of SR modified with ME, fibrinogen, and EDC: spectral representation.

FIG. 2B shows IR images at 850 cm⁻¹ vibration for thiol (left), 1400 cm⁻¹ (middle) and 1600 cm⁻¹ (right) vibrations for amide.

FIG. 3 shows Stent retriever (SR) mediated thrombectomy in a flow model: (top left) control SR before and (top right) after failed retrieval; (bottom left) fibrinogen-coated SR before and (bottom right) after successful retrieval. The blue arrows indicate the clot position.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The present disclosure provides for medical devices having a coating, medical devices for the treatment of thromboembolism and other conditions, methods of use of the medical devices, methods for putting a coating on a medical device, and the like.

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g., gorilla or chimpanzee), and rodents such as rats and mice.

As used herein, the terms “treating” and “treatment” can refer generally to obtaining a desired pharmacological and/or physiological effect. The effect can be, but does not necessarily have to be, prophylactic in terms of preventing or partially preventing a condition or outcome.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, the range includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ less than y,′ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### B. DISCUSSION

The present disclosure provides for medical devices having a coating, medical devices for the treatment of thromboembolism and other conditions, methods of use of the medical devices, methods for putting a coating on a medical device, and the like. The present disclosure is advantageous in that it can improve single-pass recanalization rates, shorten procedure times, and reduce embolic complications. In addition, the present disclosure can be advantageous since the device reduces the likelihood of slippage or incomplete extraction as well as reduces particulate shedding, which mitigates the potential for secondary embolization and downstream occlusion, a common safety concern in endovascular procedures.

In an embodiment, the present disclosure relates to medical devices comprising a substrate and a coating on at least one surface of the substrate. The coating comprises a fibrin-based material. In an embodiment, the medical device can be a stent, a stent retriever, a catheter (e.g., aspiration catheter), a metal wire (e.g., metal wire or mesh used in medical procedures), or a medical plastic tube. Fibrin is a protein in blood that can form a mesh-like structure helpful to stop bleeding and/or heal wounds and can form a stable blood clot. Fibrin can also thicken blood clots located in arteries. A stent can be a mesh tube used to prop open blood vessels. A stent retriever is a medical device that can be used to grab and pull out a blood clot from a blood vessel, restoring blood flow. A catheter is a thin, flexible tube used to drain fluids from the body.

The substrate can comprise a polymeric or metallic material, including titanium alloys, stainless steel, and plastic (e.g., thermoplastic materials or thermoset materials). The surface of the substrate can have complex geometries and be made of many different types of materials. In an aspect, the fibrin-based material can include recombinant human fibrinogen. In an embodiment, the fibrin-based material includes about 10% fibrin or more. In addition, the fibrin-based material can include about 10% or more, about 20% or more, about 30% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100%, about 10 to 90%, about 20 to 90%, about 30 to 90%, about 40 to 90%, about 50 to 90%, about 60 to 90%, about 70 to 90%, or 80 to 90% fibrin. In an embodiment, the fibrin-based material is covalently bonded to the surface of the substrate via thiol and/or amide bonding linkages. In an embodiment, the fibrin-based material is covalently bonded to the surface of the substrate by one or more bonds: S—S bonding (sulfur-sulfur bonding), thiourea bonding, thiocarbamate bonding, and thioester bonding.

In an embodiment, the fibrin-based material is configured to form a covalent bond with fibrin present in a second composition. For example, S—S bonding is formed covalently by the reaction of sulfur containing reagents (e.g., thio alcohol, thioester or thiourea) formed on the surface between the plasma modified metal or polymer with fibrinogen, stabilized through crosslinking and the fibrin clot. The second composition can be a thrombus containing an amount of fibrin. In an aspect, the thrombus includes about 10% or more, about 20% or more, about 30% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100%, about 10 to 90%, about 20 to 90%, about 30 to 90%, about 40 to 90%, about 50 to 90%, about 60 to 90%, about 70 to 90%, or 80 to 90% fibrin. Thrombus is a blood clot located inside a blood vessel or the heart. While not intending to be bound by theory, it appears that the fibrinogen coating is converted into fibrin circulating in the blood by thrombin, and that it then cross-links on fibrin-bound clot in conjunction with circulating Factor XIIIa. The medical device can be used to capture a thrombus through means of the fibrin-based material present in the coating of the medical device contacting and forming covalent bonds with the fibrin present in the thrombus.

In another embodiment, the disclosure relates to a method for treating thromboembolism, including the steps of contacting a thrombus in a subject with a medical device as described above, allowing the medical device to capture the thrombus, and then removing the thrombus from the subject. A thrombus may be located in an artery, and the medical device can extract the thrombus through the artery. In an embodiment, allowing the apparatus to capture the thrombus involves the fibrin-based material in the coating of the medical device forming covalent bonds with fibrin present within the thrombus. In an embodiment, the covalent bonds to the surface of the substrate include thiol and/or amide bonding linkages. The covalent bonds formed between the fibrin-based material in the coating of the medical device and the fibrin present within the thrombus can be S—S bonding thiourea bonding, thiocarbamate bonding, and thioester bonding. In an aspect, the method of treating thromboembolism using the medical devices described herein showed no clot aggregation, coating delamination, or particulate release.

In another embodiment, the disclosure relates to a method for forming a coating on a substrate, including the steps of modifying a substrate via low temperature plasma polymerization, exposing the substrate to a fibrinogen solution, and forming a coating on a surface of the substrate. The coating comprises a fibrin-based material. In an embodiment, the method further comprises crosslinking the fibrin-based material. The crosslinking can include exposing the fibrin-based material to carbodiimide in ethanol. Alternatively, the fibrin-based material can be exposed to glutaraldehyde or other protein-crosslinkers or coupling agents like sulfosuccinimidyl 6-[3 (2-pyridyldithio)-propionamido]hexanoate. In an embodiment, the substrate is exposed to the fibrinogen solution for about 13 to 36 hours or about 24 hours. Alternatively, the substrate is exposed to the fibrinogen solution for about 2 hours or more, about 3 hours or more, about 4 hours or more, about 5 hours or more, about 6 hours or more, about 7 hours or more, about 8 hours or more, about 9 hours or more, about 10 hours or more, about 11 hours or more, about 12 hours or more, about 13 hours or more, about 14 hours or more, about 15 hours or more, about 16 hours or more, about 17 hours or more, about 18 hours or more, about 19 hours or more, about 20 hours or more, about 21 hours or more, about 22 hours or more, about 23 hours or more, about 25 hours or more, about 26 hours or more, about 27 hours or more, about 28 hours or more, about 29 hours or more, about 30 hours or more, or any other amount of time.

In an embodiment of the above method, modifying the substrate via low temperature plasma polymerization is accomplished using a sulfur-based material. The sulfur-based material can be organo-mercaptoethanol, hydrogen sulfide, thioesters, or thioureas. In an embodiment, modifying the substrate via low temperature plasma polymerization is accomplished using the sulfur-based material in low-temperature plasma for a time-period, where temperature of the low-temperature plasma is about 25° C. to 60° C. The period of time can be about 5 minutes or more, about 10 minutes or more, about 15 minutes or more, about 20 minutes or more, about 25 minutes or more, about 30 minutes or more, about 35 minutes or more, about 40 minutes or more, about 45 minutes or more, about 50 minutes or more, about 55 minutes or more, about 60 minutes or more, about 65 minutes or more, about 70 minutes or more, about 75 minutes or more, about 80 minutes or more, about 85 minutes or more, about 90 minutes or more, about 95 minutes or more, about 100 minutes or more, about 120 minutes or more, about 180 minutes or more, about 200 minutes or more, about 240 minutes or more, about 30 minutes to about 1 hour, about 30 minutes to about 2 hours, about 1 hour to about 2 hours, and any other period of time.

In an embodiment, during modifying the substrate using low temperature plasma polymerization, organic precursors are added to the plasma. The organic precursors can be a gas or vaporized liquid such as an organic thiol, thioester, or thiourea. In this method, the substrate can comprise a polymeric or metallic material.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Cold Plasma Process Ensnares Fibrin-Rich Clots in an Adhesive Web

Advances in mechanical thrombectomy (MT) devices have reduced mortality and improved the quality of life among stroke patients. Favorable (mRS 0-2 at 90 days) post-procedure outcomes depend heavily upon the degree of recanalization. Fibrin-rich thrombi pose a major impediment to adequate (TICI 2B) clot retrieval, as their firm composition resists extraction. We describe a low-temperature plasma process for modifying stent retrievers with fibrinogen. Fibrinogen binding translates into greater efficacy in capturing fibrin-rich clots in vitro. This advance may improve MT outcomes through faster and more complete clot retrievals.

Disclosed is a cold plasma process for covalently bonding fibrinogen onto existing SRs. This method was chosen for its speed relative to conventional heat-set or ultraviolet curing, as well as an “agnostic” affinity for various surfaces, including metals and polymers.

### Methods

### Plasma Process.

The experimental arm consisted of SR (Embotrap II, Johnson & Johnson MedTech, New Brunswick, NJ) coated with recombinant human fibrinogen (Sigma-Aldrich, St Louis, MO) via organo-mercaptoethanol (ME) plasma polymerization.^{13,14} SR modification was performed using 1 mL ME (Thermo-Fisher Scientific, Waltham, MA) in low-temperature plasma (LTP) for 45 min in a plasma cleaner (Harrick Plasma, NY, USA). The 45 W setting was employed, without feed gas. Organic precursors were added in 100 μL increments per run. The evaporation rate of the ME precursor controlled the mass flow in the system. After ME modification, SRs were treated with a saturated solution of fibrinogen for 24 h. Fibrinogen crosslinking was then accomplished using 5 mM carbodiimide (EDC) in ethanol overnight.

Fourier transform infrared (FT-IR) and optical photothermal infrared (OPT-IR) characterization.

The resulting surface chemistry of the SR modification via ME plasma plus fibrinogen was analyzed by FT-IR spectroscopy using a Bruker ALPHA II spectrometer equipped with a platinum ATR module. The ATR unit features a diamond crystal for sample analysis. Prior to each measurement, the ATR crystal was cleaned with isopropanol and dried with a lint-free Kim wipe. Samples were placed directly on the ATR crystal and secured using the pressure applicator of the instrument to ensure good contact. OPT-IR spectroscopy was used for chemical mapping of functional group changes due to thiol, ME, and EDC crosslinking of fibrinogen from the characteristic thiol bending and protein amide peaks. A microscope objective is used to direct the infrared beam onto the sample and gather the signal produced. A spatial map of infrared absorption properties (distribution and con-centration) is created by tracing the beam across the entire sample.

### Clot Preparation.

Fibrin-rich clots were prepared from human subjects under an existing IRB-approved protocol according to standard techniques.^{5,15} Briefly, whole blood was drawn from healthy individuals into citrate anticoagulated tubes. Plasma, red blood cell (RBC), and buffy coat were separated by centrifugation (20 min). Plasma was pipetted out and collected, and the remaining RBC and buffy coat were mixed by inversion. 2.970 mL of plasma were treated with 3 μL of thrombin (1 NIH/mL). 0.3 mL of 5% calcium chloride solution was added, followed by 30 μL of RBC buffy coat mixture, resulting in a nearly RBC-free clot composition. The tube was rapidly mixed 5 times by inversion. This mixture was drawn into a 3 ml syringe and spun at 20 r/min overnight at room temperature to mimic the dynamic condition of a human blood vessel. Compositional analysis or comparison to patient-derived thrombi was not performed.

### Flow Model.

Clots were placed into the M1 correlate of a silastic flow model (United Biologics silicone). Silicone has been shown to demonstrate frictional clot coefficients equivalent to cerebral vasculature.¹⁶ A SR (control and fibrinogen-coated were utilized 1:1) was deployed at the thrombus, and the pump actuated to a pulsatile flow rate of 1 L and 124 pulses/min. Normal human internal carotid artery flow (<400 mL/min) and heart rate (<100 bpm) are both significantly lower. Supraphysiologic settings were chosen to accentuate the effect of weak clot integration by the SR. For each experiment, the control device was utilized first, and the same clot was then replaced into the model for another pass with the coated device.

In five tests, 500 ml of sheep plasma circulated at a temperature of 30° C., enriched with 3 mL of plasma from the human blood donor. Sheep plasma was chosen due to its physiological similarity to human plasma and its relative availability. In one test, 500 mL of sheep whole blood was used instead of plasma to assess for clot formation associated with the fibrinogen-coated SR. After 2 minutes' integration time, the SR was retracted into a guide catheter positioned at the ICA correlate. Clots dislodged from the control SR were captured in a filter basket and replaced in the “M1” segment for a second pass with the fibrinogen-coated device. Paired control and coated SR were tested 6 times; no device was used more than once in this fashion.

### Results

### Surface Characterization.

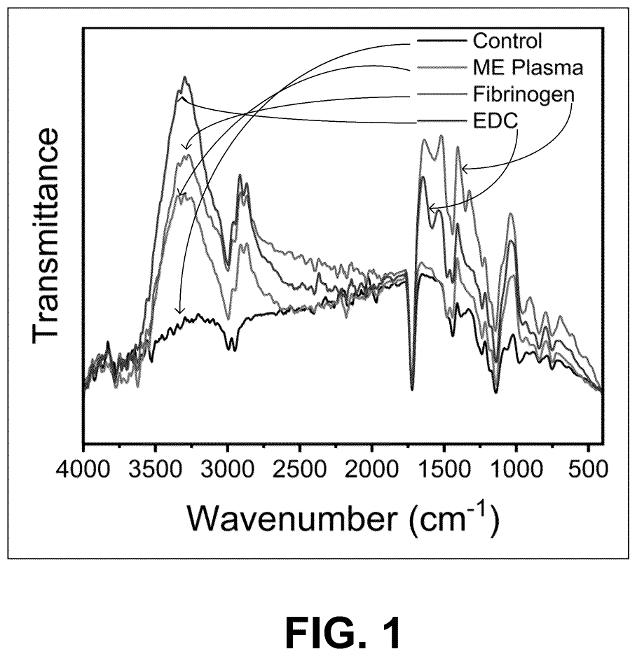

SR surface modification was characterized using FT-IR (FIG. 1). After ME modification, the retriever's characteristic vibration around 3000 cm−1 broadened due to the presence of OH groups.¹⁷ Fibrinogen binding with the ME modified retrievers led to the appearance of a peak around 1567.98 cm−1, representing amines. Finally, crosslinking of fibrinogen with EDC resulted in a shift of the amine peak.

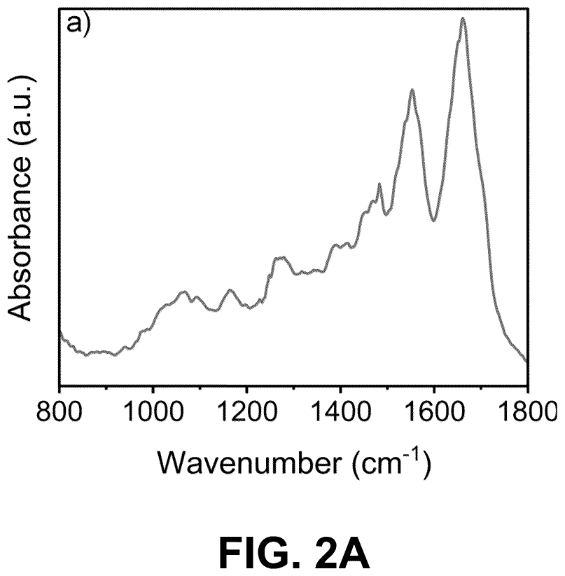

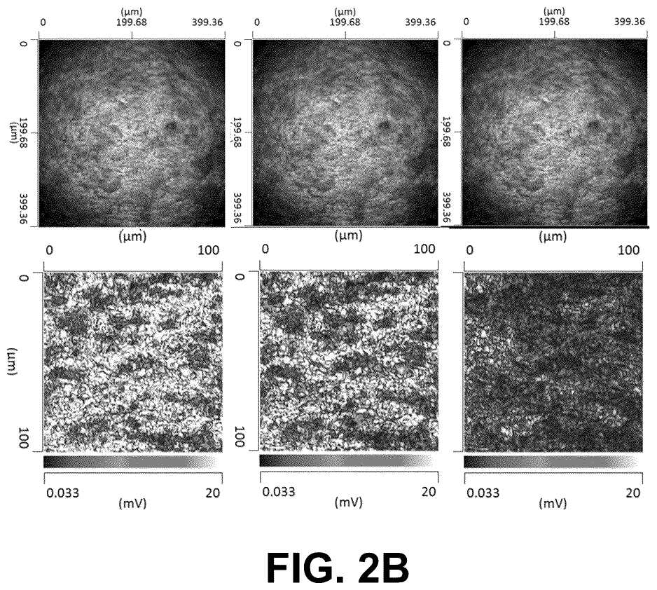

To further characterize SR surface modification, EDC cross-linked samples were subjected to (OPT-IR).¹⁸ FIGS. 2A-B show the OPT-IR spectrum (FIG. 2A) of EDC cross-linked samples. The IR mapping (FIG. 2B) was recorded for thiol bending at 850 cm−1 and amide bending vibrations at 1400 and 1600 cm−1. From the mapping, it is evident that bending vibrations of amide species are increased compared to thiol. This result is in agreement with the FTIR spectra.

### In Vitro Testing.

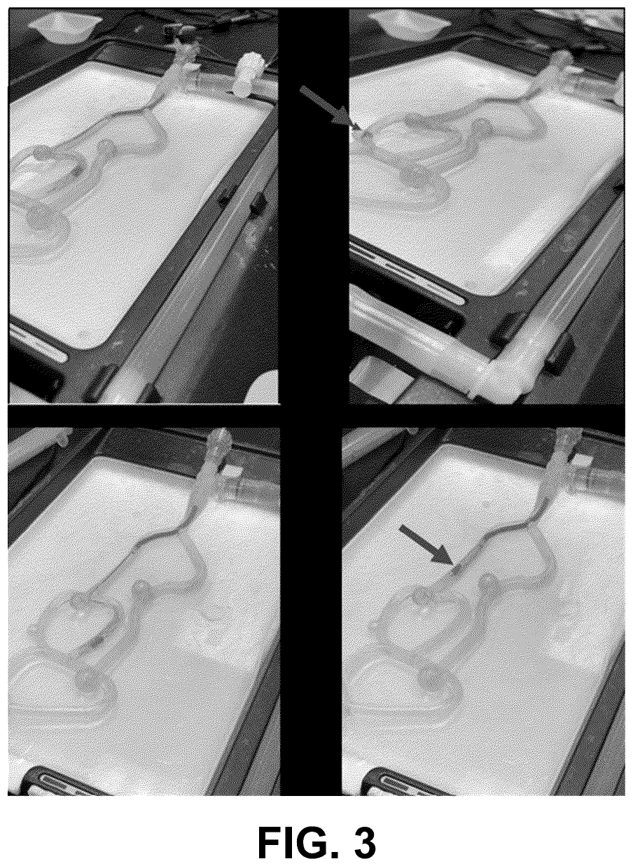

In all cases, the thrombus remained cohesive during retrieval, consistent with firm and non-friable characteristics of a fibrin-rich clot. Most clots separated from the control SR as it traversed a 90-degree curve at the “M1/ICA” junction. In 3/4 retrieval failures, the clot translated several mm distal along the SR during extraction from its initial position within the flow model, but then remained adherent until the afore-mentioned “M1/ICA” curvature, where it subsequently dislodged (FIG. 3).

No user-reported tactile differences in deployment or retrieval occurred between control and coated devices. Visual inspection of the filter basket (pore size of 125-150 μm) at the conclusion of each test revealed no new clot formation in sheep plasma or whole blood cohorts.

TABLE 1
Thrombectomy results for various clot masses. Successful
(+) and failed (−) retrievals.

| | Test | Test | Test | Test | Test | Test |
|---|---|---|---|---|---|---|
| | 1 | 2 | 3 | 4 | 5 | 6 |
| Clot weight (mg) | 19 | 22.3 | 22.6 | 24.3 | 23 | 24.1 |
| Control stent | − | + | − | − | + | − |
| retriever (SR) | | | | | | |
| Fibrinogen- | + | + | + | + | + | + |
| coated SR | | | | | | |

Coated SR successfully retrieved the clot in 6/6 tests for a “first pass” success rate of 100% (Table 1). No distal translation was observed during initial extraction from the “M1” segment, but it did occur at the “M1/ICA” curvature in one case. FIG. 3 shows the clot retrieval efficacy of a fibrinogen-coated SR compared to the control.

### Discussion.

Within the last decade, procedural thrombectomy advancements have significantly improved overall mortality and quality-adjusted life years among stroke patients.¹⁹ Adequate recanalization (TICI 2b) is the most important technical factor determining functional outcome.²⁰ Yet as refinements to MT become asymptotic, there is growing demand for novel approaches to address recalcitrant (i.e. fibrin-rich) thrombi. Fibrin cross-linking results in a firm, difficult-to-retrieve thrombus. Indiscriminate baseline imaging leaves the interventionist to suspect a fibrin-rich clot only after several failed passes.⁶

Plasma, the fourth phase of matter, represents a quasi-neutral ionized state. Non-thermal or low-temperature plasmas (LTP) are defined by their non-equilibrium thermodynamic behavior, with electrons being hotter (usually two orders of magnitude Kelvin) than ions or neutral atoms/molecules. LTPs may enhance the biocompatibility of materials by modifying their surface without affecting bulk properties such as bending moment. Additionally, LTP techniques are often greener than traditional “wet” chemical methods because they produce few bulk byproducts requiring disposal. In the present study, we used organic thiol (ME) as a plasma precursor gas to modify the SR surface, preparing it for covalent bonding with fibrinogen.

“Sticky” SR coated with various functional groups has emerged as a promising strategy. While first developed to reduce fragmentation from more easily retrieved RBC-rich clots,²¹ we present a fibrinogen layering technique for the most challenging vessel occlusions. While transglutaminase (FXIIIa) mediated fibrin cross-linking stiffens nucleating thrombi, disulfide bonds formed between cysteine residues of fibrinogen are competitive and stochastic.^{22,23} We exploit this property to enhance SR-clot interaction, leading to improved performance in vitro. We hypothesize that SR-bound fibrinogen interacts with circulating thrombin and FXIIIa to cross-link alpha and gamma chains on the clot itself. Covalent adhesion augments stent tine integration, opening up a new vulnerability among otherwise recalcitrant thrombi.

Fibrin-rich thrombi resist mechanical clot retrieval due to their firm composition. We describe a novel fibrinogen coating that enhances SR performance in vitro. The imputed mechanism involves covalently binding to the very substance—fibrin—impeding recanalization. As thrombi remain difficult to characterize with screening CT, this innovation may improve the first-pass effect across all comers.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

### REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
Ref 1 Saini V, Guada L and Yavagal D R. Global epidemiology of stroke and access to acute ischemic stroke interventions. Neurology [Internet] 2021 Nov. 16 [cited 2025 Apr. 6]; 97:57-58. Available from: https://www.neurology.org/doi/10. 1212/WNL.0000000000012781.
Ref 2 Berkhemer O A, Fransen P S S, Beumer D, et al. A randomized trial of intraarterial treatment for acute ischemic stroke. N Engl J Med 2015 Jan. 1; 372: 11-20.
Ref 3 LeCouffe N E, Kappelhof M, Treurniet K M, et al. 2B, 2c, or 3: what should be the angiographic target for endovascular treatment in ischemic stroke? Stroke 2020 June; 51:1790-1796.
Ref 4 Imahori T, Tateshima S and Kaneko N. Abstract number—169: intraprocedural angiographic sign for assessing the stent-clot interaction during mechanical thrombectomy. SVIN 2023 March; 3: e12596.
Ref 5 Gunning GM, McArdle K, Mirza M, et al. Clot friction variation with fibrin content; implications for resistance to thrombectomy. J NeuroIntervent Surg 2018 January; 10: 34-38.
Ref 6 Duffy S, McCarthy R, Farrell M, et al. Per-pass analysis of thrombus composition in patients with acute ischemic stroke undergoing mechanical thrombectomy. Stroke 2019 May; 50: 1156-1163.
Ref 7 Jolugbo P and Ariens R A S. Thrombus composition and efficacy of thrombolysis and thrombectomy in acute ischemic stroke. Stroke 2021 March; 52: 1131-1142.
Ref 8 Machi P, Jourdan F, Ambard D, et al. Experimental evaluation of stent retrievers' mechanical properties and effectiveness. J NeuroIntervent Surg 2017 March; 9: 257-263.
Ref 9 Gaertner F and Massberg S. Blood coagulation in immunothrombosis—at the frontline of intravascular immunity. Semin Immunol 2016 December; 28: 561-569.
Ref 10 Van Den Berg R, Ribó M, Arnberg F, et al. P64 NIMBUS geometric clot extractor for tough clots: SPERO study results and clot composition. In: 14th Congress of the European Society of Minimally Invasive Neurological Therapy 2022 Meeting Abstracts [Internet]. BMJ Publishing Group Ltd.; 2022 [cited 2025 Apr. 6]. p. A34.1-A34. Available from: https://jnis.bmj.com/lookup/doi/10.1136/neurintsurg-2022-ESMINT.85.
Ref 11 Haussen D C, Rebello L C and Nogueira R G. Optimizing clot retrieval in acute stroke: the push and fluff technique for closed-cell stentrievers. Stroke 2015 October; 46: 2838-2842.
Ref 12 Weafer F M, Duffy S, Machado I, et al. Characterization of strut indentation during mechanical thrombectomy in acute ischemic stroke clot analogs. J NeuroIntervent Surg 2019 September; 11: 891-897.
Ref 13 Tucker B S, Baker P A, Xu K G, et al. Atmospheric pressure plasma jet: a facile method to modify the intimal surface of polymeric tubular conduits. J Vacuum Sci Technol A: Vacuum, Surfaces, and Films 2018 Jul. 1; 36: 04F404.
Ref 14 Pillai R R, McMahan B M, Hebert L, et al. Plasma surface acti-vated carbon fiber mats for effective aerosol-mediated Mn remediation. ACS Sustain Resour Manage 2024 December 26; 1: 2602-2611.
Ref 15 Fitzgerald S T, Liu Y, Dai D, et al. Novel human acute ischemic stroke blood clot analogs for in vitro thrombectomy testing. AJNR Am J Neuroradiol 2021 July; 42: 1250-1257.
Ref 16 Elkhayyat O N, Froehler M T and Good B C. Analysis of frictional forces in experimental models of stent retriever mechan-ical thrombectomy. J Biomech 2024 February; 164: 111971.
Ref 17 Zhang X and Wang S. Voltametric behavior of noradrenaline at 2-mercaptoethanol self-assembled monolayer modified gold electrode and its analytical application. Sensors 2003 Feb. 21; 3: 61-68.
Ref 18 Karthik C, Pillai R R, Moreno G H, et al. Plasma/ozone induced PolyNaSS graft-polymerization onto PEEK biomaterial for bio-integrated orthopedic implants. JOM 2024 October; 76: 5662-5674.
Ref 19 Goyal M, Menon B K, van Zwam W H, et al. Endovascular thrombectomy after large-vessel ischaemic stroke: a meta-analysis of individual patient data from five randomised trials. Lancet 2016 Apr. 23; 387: 1723-1731.
Ref 20 Powers W J, Rabinstein A A, Ackerson T, et al. Guidelines for the Early Management of Patients With Acute Ischemic Stroke: 2019 Update to the 2018 Guidelines for the Early Management of Acute Ischemic Stroke: A Guideline for Healthcare Professionals From the American Heart Association/American Stroke Association. Stroke [Internet] 2019 December [cited 2025 Apr. 17]; 50: 374-375. Available from: https://www.ahajournals.org/doi/10.1161/STR.0000000000000 211.
Ref 21 Skarbek C, Anagnostakou V, Procopio E, et al. Development of a clot-adhesive coating to improve the performance of thrombectomy devices. J NeuroIntervent Surg 2023 December; 15: 1207-1211.
Ref 22 Blombäck B, Blombäck M, Finkbeiner W, et al. Enzymatic reduction of disulfide bonds in fibrinogen by the thioredoxin system I. Identification of reduced bonds and studies on reoxidation process. Thromb Res 1974 January; 4: 55-75.
Ref 23 Lord ST. Molecular mechanisms affecting fibrin structure and stability. ATVB 2011 March; 31: 494-499.

What is claimed is:

1. A medical device, comprising:
a substrate; and
a coating on a surface the substrate, the coating comprising a fibrin-based material, wherein the medical device is a stent, a stent retriever, a catheter, a metal wire, or a medical plastic tube.

2. The medical device of claim 1, wherein the fibrin-based material includes about 40% fibrin or more.

3. The medical device of claim 1, wherein the fibrin-based material is covalently bonded to the surface of the substrate via S—S bonding.

4. The medical device of claim 1, wherein the fibrin-based material has the characteristic to form a bond with fibrin present in a second composition.

5. The medical device of claim 4, wherein the second composition is a thrombus.

6. The medical device of claim 1, wherein the medical device is configured for capturing the thrombus.

7. A method for treating thromboembolism, comprising:
contacting a thrombus in a subject with the medical device of claim 1;
allowing the medical device to capture the thrombus; and
removing the thrombus from the subject.

8. The method of claim 7, wherein allowing the apparatus to capture the thrombus involves the fibrin-based material in the coating of the medical device forming covalent bonds with fibrin present within the thrombus.

9. The method of claim 8, wherein the covalent bonds formed between the fibrin-based material in the coating of the medical device and the fibrin present within the thrombus are S—S bonds.

10. A method for forming a coating on a substrate, comprising:
modifying a substrate via plasma polymerization;
exposing the substrate to a fibrinogen solution; and
forming a coating on a surface of the substrate, wherein the coating comprises a fibrin-based material.

11. The method of claim 10, further comprising crosslinking the fibrin-based material.

12. The method of claim 11, wherein the crosslinking includes exposing the fibrin-based material to carbodiimide.

13. The method of claim 10, wherein the substrate is exposed to the fibrinogen solution for about 12 to 36 hours.

14. The method of claim 10, wherein modifying the substrate via plasma polymerization is accomplished using a sulfur-based material.

15. The method of claim 14, wherein the sulfur-based material is organo-mercaptoethanol, hydrogen sulfide, thioesters, or thioureas.

16. The method of claim 14, wherein modifying the substrate via plasma polymerization is accomplished using the sulfur-based material in low-temperature plasma for a time-period.

17. The method of claim 16, wherein the time period is about 30 to 60 minutes.

18. The method of claim 16, wherein during modifying the substrate using plasma polymerization, organic precursors are added to the plasma.

19. The method of claim 18, wherein the organic precursors comprise an organic thiol, a thioester, or a thiourea.

20. The method of claim 10, wherein the substrate comprises a polymeric or metallic material.
